(19) United States
(12) Patent Application Publication
(10) Pub. No.: US 2024/0358607 A1
(43) Pub. Date: Oct. 31, 2024

(54) MANUFACTURING METHOD FOR SOLID PERSONAL CARE PRODUCT

(71) Applicant: Kao Corporation, Tokyo (JP)

(72) Inventors: Takahiro HIRANO, Tokyo (JP); Shohei NATSUI, Tokyo (JP); Tomoyuki MOTEGI, Tokyo (JP)

(73) Assignee: Kao Corporation, Tokyo (JP)

(21) Appl. No.: 18/768,004

(22) Filed: Jul. 10, 2024

Related U.S. Application Data
(63) Continuation of application No. PCT/JP2023/015538, filed on Apr. 19, 2023.

(30) Foreign Application Priority Data
Jun. 15, 2022 (JP) 2022-096370

(51) Int. Cl. A61K 8/02; B23C 5/10

(52) U.S. Cl. CPC A61K 8/022; B23C 5/10; A61K 2800/10

(57) ABSTRACT

A manufacturing method for a solid personal care product having a three-dimensional pattern comprises a cutting process of cutting a solid base material provided on a target object such that the base material is not penetrated. In the cutting process, the base material can be cut such that a stepped portion can be formed, where the stepped portion can include a plurality of layered stairs each having a stair width in a height direction of 0.001 mm or more and 1 mm or less, and a difference in height between a topmost portion and a bottommost portion of a surface of the base material can be 1 mm or more.

### CROSS-REFERENCE TO RELATED APPLICATION(S)

The present application is a continuation of International Application No. PCT/JP2023/015538, filed Apr. 19, 2023, which claims priority from Japanese Patent Application No. 2022-096370, filed Jun. 15, 2022, the entire content and disclosure of each is hereby incorporated by reference in entirety.

### SUMMARY

A manufacturing method according to one or more aspects of the present disclosure can be regarded as a manufacturing method for a solid personal care product having a three-dimensional pattern.

The manufacturing method according to one or more embodiments can comprise a cutting process of cutting a solid base material provided on a target object such that the base material is not penetrated.

In the manufacturing method according to one or more embodiments, in the cutting process, a layered stair having a stair width in a height direction of 0.001 mm or more and 1 mm or less can be formed.

In the manufacturing method according to one or more embodiments, a stepped portion including a plurality of the layered stairs can be formed.

In the manufacturing method according to one or more embodiments, in the cutting process, the base material can be cut such that a difference in height between a topmost portion and a bottommost portion of a surface of the base material is 1 mm or more.

### BRIEF DESCRIPTION OF THE DRAWINGS

FIG. 1 is a schematic view showing a configuration example of a cutting device according to this embodiment.

FIG. 2 is a schematic perspective view showing a configuration example of a solid personal care product.

FIG. 3 is a schematic cross-sectional view showing a configuration example of the solid personal care product.

FIG. 4 is a flowchart showing an example of a manufacturing method for the solid personal care product.

FIG. 5 is a schematic view showing an example of the manufacturing method for the solid personal care product.

FIG. 6 is a schematic view for describing the process of cutting a stepped portion.

FIG. 7 is a schematic view for describing the process of cutting along an XY plane.

FIG. 8 is a map showing heights in an example of a three-dimensional pattern.

FIG. 9 is a graph showing a profile of height along the line AA shown in FIG. 8.

FIG. 10 is a table showing the profile of height along the line AA shown in FIG. 8.

FIG. 11 is a map showing heights in another example of the three-dimensional pattern.

FIG. 12 is a graph showing a profile of height along the line BB shown in FIG. 11.

FIG. 13 is a table showing the profile of height along the line BB shown in FIG. 11.

FIG. 14 is a schematic view showing press molding using a mold as a comparative example.

### DETAILED DESCRIPTION

In a method of imparting a shape using a mold, a three-dimensional shape may be partially chipped or its corner may be rounded, and processing accuracy may be lowered. Further, as in Patent Literature 1, the processing such as removing the cosmetic layer may limit a three-dimensional expression.

An object of the present disclosure, among one or more objects, relates to a manufacturing method for a solid personal care product, which is capable of providing a solid personal care product that can have an elaborate three-dimensional pattern that can be rich in a three-dimensional effect.

Thus, according to the one or more embodiments of the present disclosure, it can be possible to provide a solid personal care product that can have an elaborate three-dimensional pattern that can be rich in a three-dimensional effect.

Hereinafter, an embodiment of the present disclosure will be described with reference to the drawings.

### [Configuration of Cutting Device]

A cutting device 100 according to an embodiment of the present disclosure can be regarded as a device that can perform a cutting process in a manufacturing process of a solid personal care product 10, for instance, that can be applicable to a human body.

In the cutting process, the cutting device 100 can cut a solid base material 12, which can be provided on a target object 11 and formed as a solid personal care product 10, so that a three-dimensional pattern 40 is formed on the surface of the base material 12. Thus, the solid personal care product 10 having the three-dimensional pattern 40 can be manufactured.

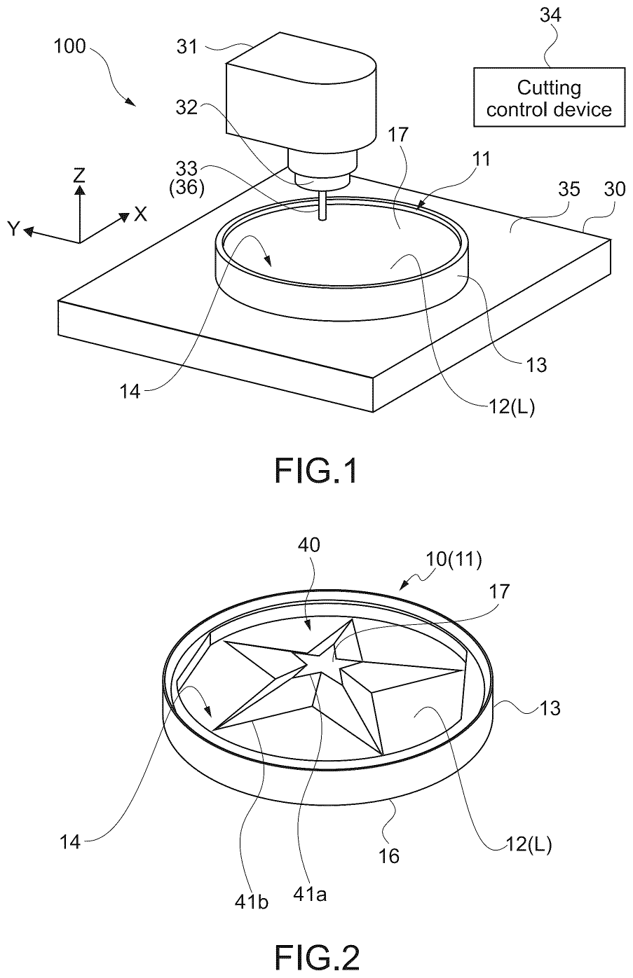

FIGS. 1 and 2 show the cutting device 100 and an exemplary embodiment of the solid personal care product 10 manufactured using the cutting device 100, respectively, according to one or more embodiments of the present disclosure.

The solid personal care product 10, which can be manufactured by the manufacturing method performed using the cutting device 100 according to one or more embodiments of the present disclosure, can be applicable to a human body by directly applying the solid personal care product 10 onto skin or by applying, spraying, or dropping liquid, which is obtained by dissolving or dispersing the solid personal care product 10 in a liquid medium such as water, onto the skin, for example. Further, the solid personal care product 10 may be one that applies an aroma to a human body or may be, for example, an aroma candle that provides a relaxing effect to a human body via an aroma. The solid personal care product 10 can be solid at one atmospheric pressure and at 20° C., for instance.

Examples of such a solid personal care product 10 can include, but are not limited to, solid cosmetics such as make-up cosmetics, solid soaps, and solid bath additives.

Examples of the make-up cosmetics can include eye shadows and foundations containing cosmetic powders, and lipsticks containing oils and pigments.

The target object 11 can be a target to be processed (work) in the manufacturing process of the solid personal care product 10. The target object 11 on which the cutting process is to be performed can include, for instance, the solid base material 12 and a container 13.

FIG. 1 schematically illustrates the target object 11 before subjected to the cutting process. In the illustrated example, the container 13, which may be regarded as a dish-like container or a container in the form of a dish, can be filled with the solid base material 12, which can be used as the target object 11.

The base material 12 can be a solid material constituting the solid personal care product 10. The base material 12 can contain powder, according to one or more embodiments of the present disclosure. Specifically, a composition L containing powder can be introduced into the container 13 and then solidified, which can be used as the base material 12.

Hereinafter, a case where the solid personal care product 10 is a solid cosmetic product made of a powder cosmetic will be described. Note, however, that the solid personal care product 10 is not limited to the above and may be other products such as solid soaps, solid bath additives, and aroma candles.

The powder cosmetic constituting the base material 12 may be either a dry cosmetic or a wet cosmetic.

In the case of dry cosmetic, the dry cosmetic can be obtained by filling the container 13 with a powdery composition and pressing a composition L to be solidified. In this case, the composition L can be made of powder such as a cosmetic. The powdery composition L may be made of one type of powder or a mixture of a plurality of types of powder.

The wet cosmetic can be obtained by supplying a composition L having fluidity to the container 13 and then solidifying the composition L. Examples of the composition L having fluidity can include: a dispersion liquid (so-called slurry) that is a mixture containing powder such as a powder cosmetic and a liquid dispersion medium; a solution obtained by dissolving various compounds such as cosmetics in a liquid solvent; a melting liquid obtained by heating and melting a cosmetic or oil alone, or a composition L containing a cosmetic. The composition L having fluidity can be solidified by absorbing and drying a volatile component in the composition L.

The container 13 can be a member, which can be regarded as a dish-like member or a member in the form of a dish, that is open on the upper side and configured so as to be capable of housing the composition L (base material 12). The container 13 can include a concave housing portion 14 that houses the base material 12 (composition L) and functions as a cosmetic tray.

The material constituting the container 13 may not be particularly limited, and for example, paper, a film, a nonwoven fabric, metal, and resin can be used. Further, the container 13 may be constituted by combining those materials.

In the illustrated example, the container 13 having a circular planar shape can be used, but the shape of the container 13 can be discretionarily set. For example, a container 13 having a polygonal shape such as a triangular or rectangle planar shape, or a container 13 having a semi-circular or elliptical planar shape may be used.

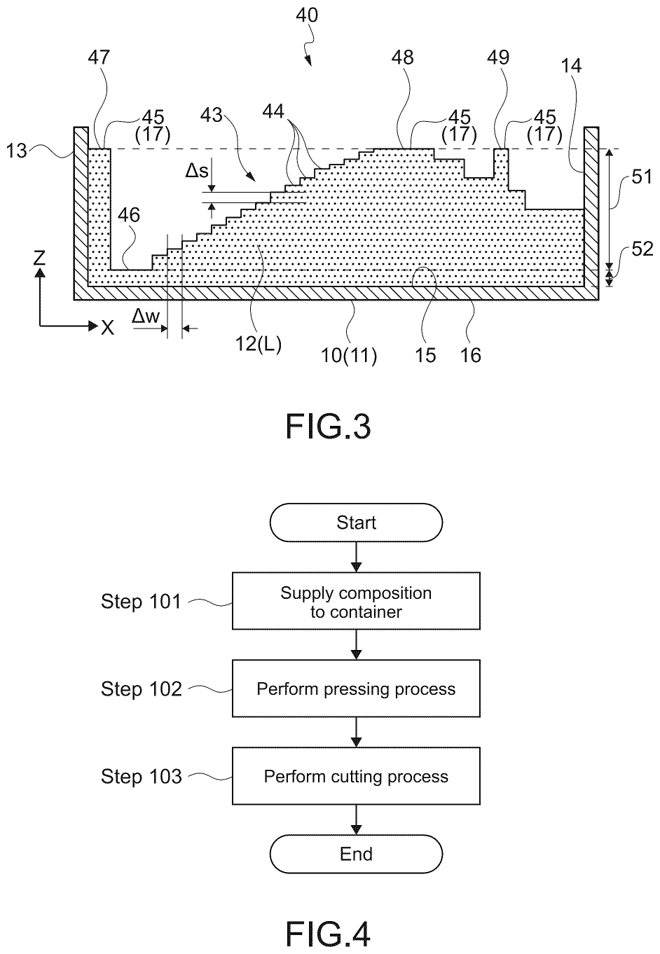

Hereinafter, the bottom surface of the housing portion 14 for housing the base material 12 (composition L) in the container 13 will be referred to as a container bottom surface 15 (see FIG. 3). Further, the surface of the container 13 on the side opposite to the side on which the housing portion 14 is provided will be referred to as a container back surface 16. The container bottom surface 15 and the container back surface 16 can be the upper surface and the lower surface of a portion constituting the bottom of the housing portion 14 and can be constituted as the surfaces parallel to each other.

Hereinafter, a height direction of the base material 12 (composition L) formed in the container 13 will be referred to as a Z direction. The Z direction is a direction orthogonal to the container bottom surface 15 (container back surface 16).

Further, a plane orthogonal to the Z direction will be referred to as an XY plane, and directions orthogonal to each other on the XY plane will be referred to as an X direction and a Y direction. For example, the container bottom surface 15 (container back surface 16) can be a surface parallel to the XY plane. In this embodiment, the direction (X direction or Y direction) along the XY plane can correspond to a plane direction orthogonal to the height direction.

The base material 12 can be a powder cosmetic on the target object 11, for instance, pressed on the target object 11. The base material 12 can be pressed to have a planar shape on the target object 11, but it is not limited to such shape. The base material 12, on which a mark such as a logotype or a predetermined three-dimensional shape can be formed, for instance, by pressing, may be subjected to cutting processing. As will be described later, in the manufacturing method according to this embodiment, after a powder cosmetic as the composition L is supplied to the container 13, a pressing process of pressing the composition L can be performed. A cutting process may then be performed after the pressing process.

In the pressing process, the composition L can be pressed such that the surface of the composition L, which can be exposed as viewed from the upper side of the container 13, becomes flat. Therefore, the surface of the base material 12 of the target object 11 before subjected to cutting can be regarded as a flat pressed surface 17 without irregularities.

For example, in the target object 11 shown in FIG. 1, the base material 12 having the flat pressed surface 17 can be formed inside the container 13. The pressed surface 17 may be formed parallel to the XY plane (container bottom surface 15 or container back surface 16), according to one or more embodiments of the present disclosure.

The cutting device 100 can cut the solid base material 12 provided on the target object 11.

The cutting device 100 can include a stage 30, a support unit 31, a drive unit 32, a cutting tool 33, and a cutting control device 34. The cutting device 100 can be configured to cut the base material 12 by using the cutting tool 33. Further, the cutting device 100 may be provided with, for example, a suction unit that suctions the base material 12 cut off.

The stage 30 can be a placing table on which the target object 11 is placed. The stage 30 can be, for example, a member, which may be a flat plate-like member or a member that is flat and in the form of a plate, and one main surface of the stage 30 can serve as a placing surface 35 on which the target object 11 (container 13) is placed. The target object 11 can be fixed to the placing surface 35 of the stage 30 by using a fixing member. For the fixing member, for example, a pressure-sensitive adhesive tape, a jig, or the like that fixes the placing surface 35 and the container 13 to each other may be used.

The stage 30 can be configured as, for example, a shuttle of a magnetic floating linear transport device. In this case, the stage 30 can function as a transport mechanism. In addition to those above, the stage 30 may be configured as a biaxial stage that moves in the XY directions or a triaxial stage that moves in the XYZ directions.

The support unit 31 can be regarded as a member for supporting the drive unit 32 and the cutting tool 33, and can support the drive unit 32, to which the cutting tool 33 is attached, for instance, directly above the stage 30. In the example shown in FIG. 1, the support unit 31 can be configured as a cantilevered structural member, which can be connected to a column provided at a position being in no contact with the stage 30 and can support the drive unit 32 at a tip portion thereof. A specific configuration of the support unit 31 is not limited. For example, a bridge-shaped support unit 31 whose both ends are connected to columns to support the drive unit 32 at the central portion thereof, or the like may be used.

The drive unit 32 can rotationally drive the cutting tool 33. For example, the drive unit 32 can be a motor including a fixture (chuck or the like) for fixing the cutting tool 33. Alternatively, a rotating member provided with a fixture may be used as the drive unit 32. In this case, the rotating member (fixture) can be connected to a motor via a pulley, a gear, or the like.

The rotation axis of the drive unit 32 (cutting tool 33) can be set so as to be orthogonal to the placing surface 35 of the stage 30.

The cutting tool 33 can cut the base material 12 while being in contact with the base material 12.

Specifically, the cutting tool 33 can include a rotary blade 36. In this embodiment, the base material 12 can be cut by the rotary blade rotated by the drive unit 32. An end mill may be used as such a cutting tool 33. Note that a drill may also be used as the cutting tool 33.

The cutting control device 34 can have a hardware configuration for a computer such as a CPU or a memory (RAM, ROM) and can control the operation of the cutting device 100. For example, the CPU can load a control program (e.g., a basic program) for the cutting device 100, which can be stored in a storage unit, into the RAM and can perform or execute the program, so that each unit (stage 30, drive unit 32, or the like) of the cutting device 100 operates.

The cutting control device 34 can read a cutting operation program as a subroutine of the basic program, for example. The cutting operation program can be a program that causes the cutting device 100 to perform a cutting operation for forming a three-dimensional pattern 40. For the cutting operation program, for example, a numerical control (NC) program described using a G-code or the like may be used. In addition, any form of program capable of causing the cutting device 100 to operate may be used.

In the cutting process, the cutting device 100 can cut the base material by relatively moving the cutting tool 33 for cutting the base material 12 while being in contact with the base material 12, and the target object 11. This can allow highly accurate three-dimensional processing using the cutting tool 33.

Specifically, the cutting device 100 can be configured to be capable of relatively moving the cutting tool 33 and the target object 11 along the X direction, the Y direction, and the Z direction. This can be regarded as a configuration in which, for example, the cutting tool 33 and the stage 30 can be relatively moved in two directions orthogonal to each other within the placing surface 35 of the stage 30 and in a direction orthogonal to the placing surface 35.

The cutting tool 33 (support unit 31) may be moved in the XYZ directions, where the position of the stage 30, on which the target object 11 can be placed, can be fixed.

Further, the stage 30 on which the target object 11 can be placed may be moved in the XYZ directions, where the position of the cutting tool 33 (support unit 31) can be fixed.

Further, both the cutting tool 33 and the stage 30 may be moved. In this case, for example, a configuration in which the cutting tool 33 can be moved in the Z direction and the stage 30 can be moved in the XY directions can also be possible.

In addition, the method of relatively moving the cutting tool 33 and the target object 11 may not be particularly limited.

The blade diameter of the rotary blade 36 provided to the cutting tool 33 will be described. The blade diameter of the rotary blade can be, for example, a dimension in a radial direction that is set according to the shape or type of the rotary blade. For example, in a square-type end mill in which the rotation trajectory of the rotary blade is cylindrical, the blade diameter of the rotary blade can be the diameter of the rotary blade. Further, in an end mill in which the shape of an outer peripheral blade in the rotary blade is tapered, the blade diameter of the rotary blade can be the diameter of the tip of the rotary blade. Further, in an end mill in which the tip of the rotary blade is of a ball type, the blade diameter of the rotary blade can be, for example, the diameter of the ball. Further, in an end mill for V-groove processing in which the tip of the rotary blade is formed into a conical shape, the blade diameter of the rotary blade can be, for example, a large diameter of the tip portion (maximum diameter of rotary blade). In short, regarding a tool whose diameter changes, a portion that actually comes into contact with the base material 12 (actual cutting diameter) can serve as the blade diameter of the rotary blade. Further, if the cutting tool 33 is a drill, the blade diameter of the rotary blade can be, for example, the diameter of the drill.

The blade diameter of the rotary blade 36 provided to the cutting tool 33 can be 0.1 mm or more and 5 mm or less, as an example. The blade diameter of the rotary blade 36 can be discretionarily set within the above range, for example.

Of those, for example, use of a rotary blade 36 having a blade diameter of 0.1 mm or more and 2 mm or less can make it possible to easily achieve a narrow groove width and/or to achieve a highly fine three-dimensional pattern 40.

Further, for example, use of a rotary blade 36 having a blade diameter of 2 mm or more and 5 mm or less can make it possible to improve a processing speed of the cutting processing.

Note that the blade diameter of the rotary blade 36 may not be particularly limited. For example, a cutting tool 33 including a rotary blade 36 having any blade diameter may be used according to the shape of a three-dimensional pattern 40, a requested processing speed, or the like.

### [Configuration of Solid Personal Care Product]

After the cutting process is performed, as shown in FIG. 2, a three-dimensional pattern 40 can be formed on the base material 12 constituting the target object 11. The target object 11, on which the three-dimensional pattern 40 can thus be formed, can be a solid personal care product 10 having the three-dimensional pattern 40.

The three-dimensional pattern 40 may be, for example, various characters such as Japanese characters including hiragana and katakana, alphabets, Arabic numerals, Roman numerals, and foreign characters; straight lines and curves, and figures and geometric shapes formed of combinations thereof; and symbols, colors, patterns, or patterns having combined shapes thereof.

The three-dimensional pattern 40 can include a star mark having five vertices, such as shown in FIG. 2. Here, a small star 41a and a star 41b larger than and similar to the star 41a can be disposed such that their centers coincide with each other in plan view. The star 41a can be disposed at a higher position than the position at which the contour of the star 41b is formed. Stepped slopes can be formed, around the contour of the star 41b, on both sides of each ridge line connecting the corresponding vertices of the stars 41a and 41b.

The example illustrated in FIG. 2 can be regarded as an exemplary three-dimensional pattern 40, and any three-dimensional pattern 40 can be formed in the cutting process.

Hereinafter, the solid personal care product 10 (target object 11) having the three-dimensional pattern 40 will be described with reference to FIG. 3.

The solid personal care product 10 can include the solid base material 12 on which the three-dimensional pattern 40 can be formed, and the container 13 that can house the base material 12. The container 13 may be one that can be used as the target object 11. Further, a portion cut in the cutting process may be left as it is in the base material 12. Thus, at least a part of the three-dimensional pattern 40 can include a cut portion (cut surface or the like).

In the cutting process, the solid base material 12 provided on the target object 11 can be cut so as not to penetrate the base material 12.

In other words, the cutting by the cutting device 100 can be performed such that the container bottom surface 15 of the container 13 that houses the base material 12 is not exposed. This can be achieved by controlling the cutting device 100, for example, such that the Z position of the tip of the cutting tool 33 is located higher than the Z position of the container bottom surface 15.

As described above, since the cutting operation such as penetrating the base material 12 may not be performed, as shown in FIG. 3, the container bottom surface 15 of the container 13 may not be exposed in the solid personal care product 10.

This can make it possible to leave the base material 12, which can be a powder cosmetic, on the entire surface of the container 13, for instance. Further, since the container bottom surface 15 may not be cut by the cutting tool 33, a situation in which a member of the container 13 is mixed into the cosmetic can be avoided.

In the cutting process, a stepped portion 43 including a plurality of layered stairs each having a stair width Δs in the height direction (Z direction) of 0.001 mm or more and 1 mm or less can be formed.

In the stepped portion 43, for example, layer surfaces 44 parallel to the XY plane may be disposed in layers while changing height. In other words, the stepped portion 43 can include a plurality of layer surfaces 44 orthogonal to the Z direction. A difference in the Z position between the adjacent layer surfaces 44 (difference in height) can be regarded as the stair width Δs. The stair width Δs may be constant or may be different for each layer. Setting the stair width Δs in the range described above can make it possible to accurately achieve various three-dimensional patterns 40.

For example, as shown in FIG. 3, in the three-dimensional pattern 40, a stepwise slope can be formed by the stepped portion 43.

For example, from the viewpoint of forming a smooth slope, the stair width Δs can be preferably 0.2 mm or less, more preferably 0.1 mm or less, and even more preferably 0.05 mm or less. Further, for example, from the viewpoint of improving the processing speed for the stepped portion 43, the stair width Δs can be preferably 0.1 mm or more, more preferably 0.2 mm or more, and even more preferably 0.3 mm or more.

In addition, the stair width Δs may not be limited and may be appropriately set according to the design of the three-dimensional pattern 40, a requested processing speed, or the like, as examples.

A width Δw in the plane direction (XY plane direction) of a layered stair constituting the stepped portion 43 can be preferably 0.001 mm or more and 1 mm or less. The width Δw of the stair in the plane direction can be regarded as a width of the layer surface 44 in the plane direction. The width Δw may be constant, may be different for each position on the XY plane, or may be different for each layer. Setting the width Δw in the range described above can make it possible to accurately achieve various three-dimensional patterns 40.

In the cutting process, the base material 12 can be cut such that the difference in height between a topmost portion 45 and a bottommost portion 46 of the surface of the base material 12 is 1 mm or more, as an example range.

The topmost portion 45 can be a portion located at the highest Z position on the surface of the base material 12. For example, in an uncut portion, the pressed surface 17 can remain as it is, and the Z position thereof can be higher than that of a cut portion. Therefore, if the pressed surface 17 remains, that portion can be the topmost portion 45. Further, if the pressed surface 17 does not remain, it can mean that the cutting has been performed on the entire surface of the container 13. In this case, the portion at the highest Z position can be the topmost portion 45.

The bottommost portion 46 can be a portion located at the lowest Z position on the surface of the base material 12. In other words, the bottommost portion 46 can be a surface portion of the base material 12, which has been cut most deeply from the pressed surface 17.

In the example shown in FIG. 3, in the stepped portion 43 whose height is increasing toward right, the lowest portion on the left side can be the bottommost portion 46. Further, a circumferential portion 47 brought into contact with the inner wall of the container 13 and located on the left side of the bottommost portion 46 can be an uncut portion and is a topmost portion 45. Further, a top portion 48, which can be highest on the right side of the stepped portion 43, and a convex portion 49 on the right side thereof may not be cut, and thus they can be regarded as topmost portions 45.

In the solid personal care product 10, the difference in height between the bottommost portion 46 and the topmost portion 45 can be set to 1 mm or more, for instance, so that the difference in height can be certainly perceived. As a result, a three-dimensional pattern 40 that is rich in a three-dimensional effect can be achieved.

Further, in the cutting process according to one or more embodiments of the present disclosure, a narrow-width shape, a tall shape, a deep shape, and/or the like can be easily achieved as compared to the case of using a mold or the like. In the illustrated example, the relatively tall convex portion 49 with a narrow width can be formed. This can make it possible to achieve an elaborate pattern with a difference in height.

Here, the porosity of the solid personal care product 10 will be described.

The porosity can be, for example, a proportion of voids between powders in the composition L containing powder. For example, a hollow portion located between powders can be regarded as a void. Further, a portion that is located between powders and is filled with a binder such as oils and fats may be included in the void. Additionally or alternatively, in a cross section, a portion other than the portion occupied by powder larger than a predetermined particle size may be regarded as a void.

An X-ray CT can be used to analyze a tomographic image obtained by irradiating the base material with an X-ray to calculate a portion to be regarded as a void, so that the area of the void portion per unit area can be calculated as a porosity, for instance. In addition, the method of calculating the porosity may not particularly limited.

In the solid personal care product 10, it may be preferable that a porosity V1 of the base material 12 in a first region 51 located between the height position of the topmost portion 45 and the height position of the bottommost portion 46 be substantially the same as a porosity V2 of the base material 12 in a second region 52 located at a height position lower than the height position of the bottommost portion 46. For example, the solid personal care product 10 may be constituted in the range in which V1/V2=1±0.1.

As described above, in the target object 11 to be the solid personal care product 10, a flat pressed surface 17 can be formed over the entire surface of the container 13. Through this process, the base material 12 (composition L) housed in the container 13 can be uniformly pressed, and the porosity of the base material 12 in the container 13 can be made uniform.

Further, in the cutting process, three-dimensional processing of the base material 12 can be possible almost without changing the porosity of the inside of the base material 12. Hence, the porosity in the first region 51 and the porosity in the second region 52 can be substantially the same.

Such a configuration can make it possible to achieve a solid personal care product, which may have no deviation in the density of the powder cosmetic and can provide a stable amount of powder cosmetic taken or the like throughout its use.

The hardness of the base material 12 can be 0.1 N or more and 100 N or less, for instance.

For example, from the viewpoint of making the cut three-dimensional shape less likely to collapse and achieving fine processing, the hardness of the base material 12 can be preferably 1 N or more, and more preferably 3 N or more. Further, from the viewpoint of suppressing chipping, damage, or the like of the base material 12 during cutting, the hardness of the base material 12 can be preferably 50 N or less, and more preferably 30 N or less.

The hardness of the base material can be measured using a rheometer, a force gauge, a hardness meter, or the like. For example, the hardness of the base material 12 can be calculated by mounting a jig, which has a contact portion with the surface of the base material of 42 mm, on a rheometer (CR-100 manufactured by Sun Scientific Co., Ltd.) and measuring the load when the jig is pushed 1 mm from the surface of the base material.

As described above, the base material 12 csn contain powder (powder cosmetic). The mean particle size of the powder can be 0.01 μm or more and 1 mm or less, for instance.

For example, from the viewpoint of adjusting optical properties such as coloring power, brightness, and saturation, the mean particle size of the powder can be preferably 0.05 μm or more, and more preferably 0.1 μm or more.

Further, from the viewpoint of suppressing chipping or the like during cutting and of ensuring processing accuracy, the mean particle size of the powder can be preferably 0.5 mm or less, and more preferably 0.3 mm or less.

The mean particle size can be regarded as a volume cumulative particle size D50 at a cumulative volume of 50 vol. % measured by a laser diffraction/scattering particle size distribution measurement device. Note that if the mean particle size of the powder is measured from a final product, first, the product can be dissolved in water or oil, and a binder component can be dissolved to disperse the particles in the solvent. Subsequently, the particle size distribution of the solid object can be measured using a particle size distribution meter, and the resultant volume cumulative particle size D50 obtained can be defined as the mean particle size.

### [Manufacturing Method for Solid Personal Care Product]

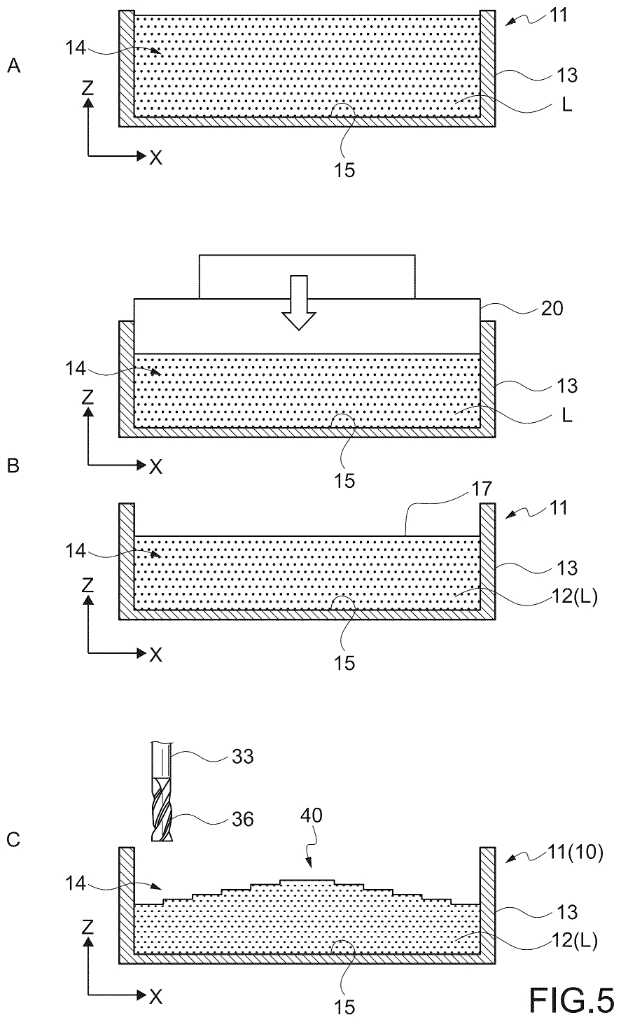

The manufacturing method for the solid personal care product 10 will be described with reference to FIGS. 4 and 5.

First, a powder cosmetic as the composition L can be supplied to the container 13 (Step 101). As shown in FIG. 5A, the composition L can be supplied, for instance, so as to fill the housing portion 14 of the container 13.

If the composition L is a dry cosmetic, for example, a predetermined amount of the composition L can be supplied to the housing portion 14, for instance, from a hopper disposed above the container 13. Further, if the composition L is a wet cosmetic, for example, a slurry-like composition L can be supplied to the housing portion 14, for instance, from a MOHNO PUMP (registered trademark) disposed above the container 13. The method of supplying the composition L to the container 13 may not be particularly limited.

Next, a pressing process of pressing the composition L supplied to the container 13 can be performed (Step 102). Here, as shown in FIG. 5B, for instance, the composition L can be pressed from above the housing portion 14 using a press head 20, and a pressed surface 17 can be finally formed. The pressed surface 17 may typically be planar.

For example, main pressing for solidifying the composition L can be performed on the composition L, with which the container 13 is filled in Step 101. After the main pressing, pressing can be performed using the press head 20 including a flat mold for finishing, and a flat pressed surface 17 can be formed.

Note that the flat pressed surface 17 may be formed by the main pressing without performing the pressing for finishing. In addition, preliminary pressing or the like may be performed before the main pressing. By such a pressing process, the target object 11 including the solid base material 12 having the flat pressed surface 17 can be formed.

Next, a cutting process of cutting the solid base material 12 can be performed (Step 103). Here, as shown in FIG. 5C, for instance, the solid base material 12 can be cut by the cutting device 100 including the cutting tool 33, and a three-dimensional pattern 40 can be formed on the surface of the base material 12.

In the cutting process, as described above, the solid base material provided on the target object can be cut so as not to penetrate the base material. Therefore, the base material 12 can be cut in the range in which the bottom (container bottom surface 15) of the container 13 may not be exposed.

In the cutting process, the three-dimensional pattern 40 can be provided to the base material 12 such that at least a part of the surface of the base material 12 cut in the cutting process can be included in the surface of the final product. In other words, at least a part of the surface of the cut base material 12 can be used as the surface of the final product as it can be without any change on the surface. Specifically, at least a part of the surface of the final product can include a cut surface that has been cut by the cutting tool 33. The cut surface described herein may not include a surface decorated by pressing or the like after the cutting process, for example. Note that the surface of the final product may include an uncut pressed surface 17. For example, in FIG. 3, the surface (e.g., bottommost portion 46, stepped portion 43, etc.) other than the uncut pressed surface 17 (circumferential portion 47, top portion 48, and convex portion 49) can be a cut surface that is to be the surface of the final product.

Further, in the manufacturing method for the solid personal care product, typically, the pressing process may not be performed after the cutting process. In this case, for example, the three-dimensional pattern 40 formed by the cutting process can be used as the pattern of the product without any change. Note that, after the cutting process, the base material 12 may be partially decorated by a method other than pressing. For example, laser processing or coating processing of a composition with a dispenser may be performed on a part of the base material 12. Even in such a case, the three-dimensional pattern 40 formed by cutting can be left on the surface of the final product.

In such a manner, use of the cut surface as the surface of the final product can eliminate the need to perform pressing process or the like after cutting. Further, also in the pressing process performed before the cutting process, for example, it may only be necessary to perform pressing using a flat mold or the like for the main pressing, and a decorative mold or the like may become unnecessary.

For example, the target object 11 on which the three-dimensional pattern 40 is provided by the cutting process can be the solid personal care product 10 having the three-dimensional pattern 40 as it is. Note that the target object 11 provided with the three-dimensional pattern 40 may be subjected to surface treatment or the like for suppressing peeling or the like of the base material 12.

### [Cutting of Stepped Portion]

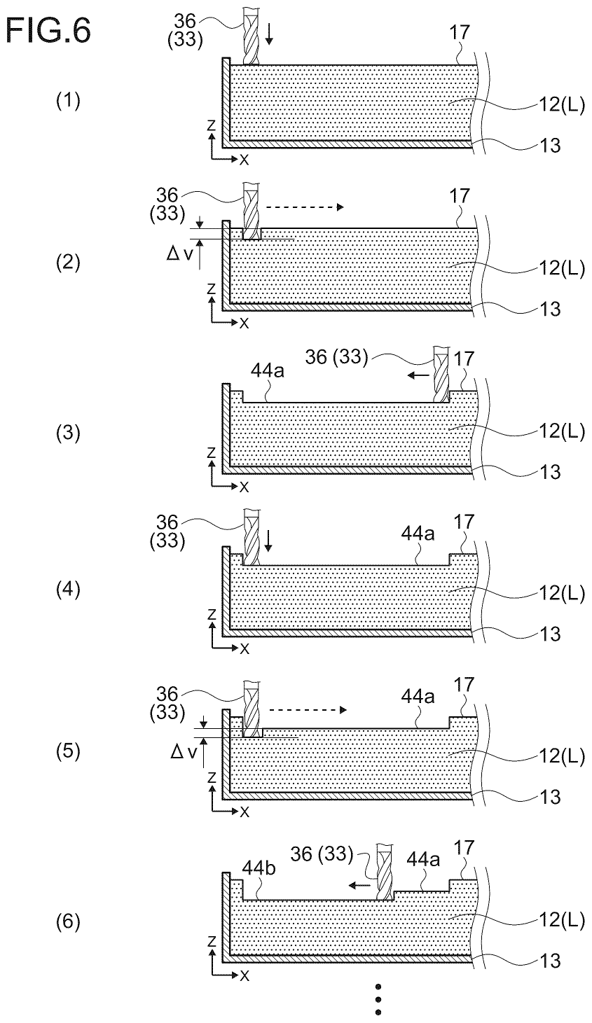

The process of cutting out the stepped portion 43 will be described with reference to FIG. 6.

In this embodiment, in the cutting process, it may be preferable to alternately repeat a first step of moving the cutting tool 33 by a predetermined amount along the height direction and a second step of moving the cutting tool 33 in a plane direction orthogonal to the height direction, to form the stepped portion 43.

In the cutting device 100, the first step can be a step of moving the cutting tool 33 in the Z direction. This can be the step of moving the cutting tool 33 along the rotation axis of the cutting tool 33.

Further, in the cutting device 100, the second step can be a step of moving the cutting tool 33 along the XY plane. This can be the step of moving the cutting tool 33 in a radial direction about the rotation axis of the cutting tool 33.

The operation of the cutting tool 33 when cutting out a layered stepped portion 43 is schematically illustrated in (1) to (6) of FIG. 6. Here, it can be assumed that cutting is started from the pressed surface 17.

In (1) of FIG. 6, at a cutting start position, the cutting tool 33 can be lowered by a predetermined amount Δv along the Z direction from the pressed surface 17 to be cut (first step). Δv can be the cut amount in the Z direction with respect to the base material 12. In the process of cutting out the stepped portion 43, Δv can correspond to the stair width Δs of the stepped portion 43. Here, a state in which the cutting tool 33 is moved along the Z direction is schematically illustrated by the solid arrow.

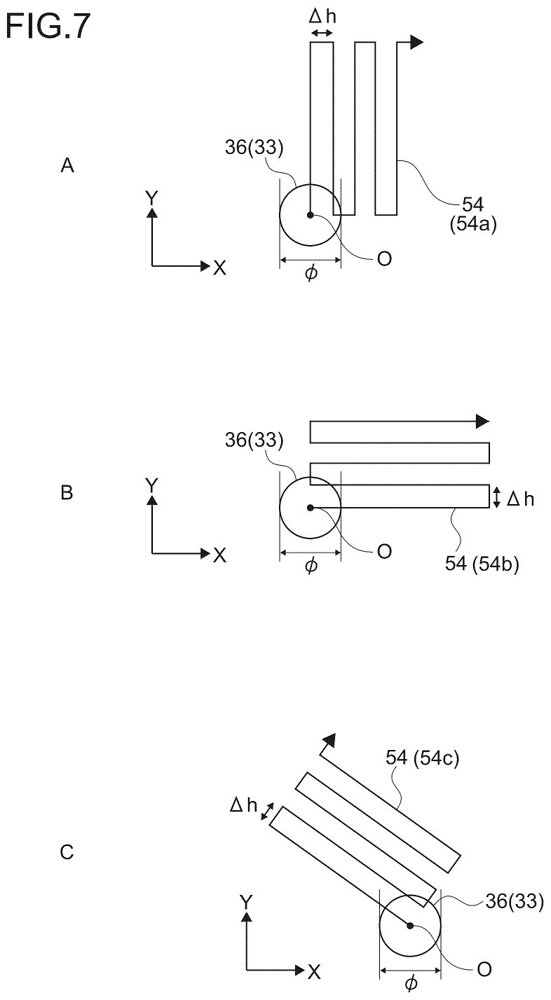

In (2) of FIG. 6, a first layer surface 44a can be formed by moving the cutting tool 33 along the XY plane with the cutting tool 33 cutting into the pressed surface 17 by Δv (second step). Here, a state in which the cutting tool 33 is moved along the XY plane is schematically illustrated by the dotted arrow. Note that the cutting along the XY plane will be described later with reference to FIG. 7.

In (3) of FIG. 6, the cutting along the XY plane for forming the first layer surface 44a can be completed. In this case, the cutting tool 33 can be moved to a cutting start position for a second layer surface 44b constituting the next layer.

In (4) of FIG. 6, at the cutting start position for the second layer surface 44b, the cutting tool 33 can be lowered by a predetermined cut amount Δv along the Z direction from the first layer surface 44a (first step). Note that the value of Δv need not be the same as that of (2).

In (5) of FIG. 6, the second layer surface 44b can be formed by moving the cutting tool 33 along the XY plane with the cutting tool 33 cutting into the first layer surface 44a by Δv (second step). Note that when the second layer surface 44b is formed, the first layer surface 44a formed in (2) may also be partially cut. Therefore, the processing of (5) can be regarded as processing of forming the second layer surface 44b and determining the planar shape of the first layer surface 44a.

In (6) of FIG. 6, the cutting along the XY plane for forming the second layer surface 44b can be completed. In this case, the cutting tool 33 can be moved to a cutting start position for the next layer.

(1) to (3) of FIG. 6 can be regarded as the process of cutting by a predetermined cut amount in the height direction using the cutting tool 33 and moving the cutting tool 33 on the XY plane to perform cutting, which can be regarded as the process of forming one layer (first layer surface 44a). Further, (4) to (6) of FIG. 6 can be regarded as the process of, after forming one layer, further cutting by a predetermined cut amount in the height direction using the cutting tool 33 and moving the cutting tool 33 on the XY plane to perform cutting, which is the process of forming the next layer (second layer surface 44b). As described above, the processes of forming the layer surfaces 44 can be repeated, so that the stepped portion 43 including a plurality of layered stairs can be formed.

In the example shown in FIG. 6, the layer surfaces 44 can have been sequentially formed from a higher Z position. In other words, stairs can have been sequentially formed from a higher Z position. The present disclosure is not limited to this, however. For example, stairs may be sequentially formed from a lower Z position. In this case, for example, a layer surface 44 at a deepest position can be formed first, and then other layers sequentially. In addition, it can also be possible to form a layer surface 44 at an intermediate position first, and then form an upper layer surface 44 and a lower layer surface 44.

In FIG. 6, an example in which the stepped portion 43 can have been directly processed from the uncut pressed surface 17 has been described. The present disclosure is not limited to this, however. For example, it may also be possible to roughly cut the base material 12 first to form an outline shape of the stepped portion 43, and then cut out the shape of the final stepped portion 43. In this case, for example, at the time of cutting out the outline shape, the area to be cut on the XY plane can be greatly reduced. Thus, for example, the processing time required for the second step can be greatly shortened, which can make it possible to shorten the overall processing time.

### [Cut Amount Δv in Height Direction]

In the cutting process, the cut amount Δv with respect to the base material 12 in the height direction (Z direction) of the cutting tool 33 can be 0.001 mm or more and 1 mm or less, for example.

The cut amount Δv in the Z direction can be the depth of the base material 12 to be cut along the XY plane. Setting the cut amount Δv in the range of the upper limit or below can make it possible to avoid a situation in which the base material 12 is broken.

As Δv becomes smaller, an elaborate three-dimensional shape including finer stairs in the Z direction can be processed. On the other hand, as Δv becomes larger, the processing time required for cutting can be shortened.

For example, from the viewpoint of achieving elaborate processing with a small stair width, the cut amount Δv in the Z direction can be preferably 0.15 mm or less, and more preferably 0.1 mm or less.

Further, for example, from the viewpoint of shortening the processing time, the cut amount Δv in the Z direction can be preferably 0.1 mm or more, and more preferably 0.2 mm or more.

### [Cutting Along XY Plane]

The process of cutting the base material 12 along the XY plane will be described with reference to FIG. 7. This process can be applicable to, for example, the second step in FIGS. 6 ((2) and (5) of FIG. 6).

FIG. 7A, FIG. 7B, and FIG. 7C each schematically illustrate a trajectory 54 of the cutting tool 33 on the XY plane, according to one or more embodiments of the present disclosure. The trajectory 54 of the cutting tool 33 can be a path through which the rotation axis O of the cutting tool 33 passes.

Further, Δh illustrated in each diagram can be regarded as a cut amount in the XY plane direction with respect to the base material 12. Δh may be set to be smaller than the diameter φ of the rotary blade 36 of the cutting tool 33, according to one or more embodiments of the present disclosure. Therefore, in the cutting along the XY plane, the base material 12 can be cut with a width of Δh.

A trajectory 54a shown in FIG. 7A can be regarded as an example of the trajectory 54 that repeats cutting along the Y direction. In this case, after cutting along the Y direction, the cutting tool 33 can be moved along the X direction by the cut amount Δh. Subsequently, the cutting along the Y direction can be resumed with the cutting tool 33 being shifted by Δh in the X direction.

In such a manner, the trajectory 54a for cutting along the Y direction and cutting with a shift along the X direction can be used, for example, when a region or the like extending along the Y direction is cut.

A trajectory 54b shown in FIG. 7B can be regarded as an example of the trajectory 54 that repeats cutting along the X direction. In this case, after cutting along the X direction, the cutting tool 33 can be moved along the Y direction by the cut amount Δh. Subsequently, the cutting along the X direction can be resumed with the cutting tool 33 being shifted by Δh in the Y direction.

In such a manner, the trajectory 54b for cutting along the X direction and cutting with a shift along the Y direction can be used, for example, when a region or the like extending along the X direction is cut.

A trajectory 54c shown in FIG. 7C can be regarded as an example of the trajectory 54 that repeats cutting along a first direction inclined with respect to the X direction and the Y direction. In this case, after cutting along the first direction, the cutting tool 33 can be moved along a second direction orthogonal to the first direction on the XY plane by the cut amount Δh. Subsequently, the cutting along the first direction can be resumed with the cutting tool 33 being shifted by Δh in the second direction. The first direction (second direction) can be discretionarily set.

In such a manner, the trajectory 54c for cutting along the first direction and cutting with a shift along the second direction can be used, for example, when a region or the like disposed to be inclined with respect to the X direction or the Y direction is cut.

### [Cut Amount Δh in Plane Direction Orthogonal to Height Direction]

In the cutting process, the cut amount Δh with respect to the base material 12 in the plane direction (direction along XY plane) orthogonal to the height direction (Z direction) of the cutting tool 33 can be 0.001 mm or more and 1 mm or less, for instance.

The cut amount Δh along the XY plane can be the width of the base material 12 to be cut on the XY plane. Setting the cut amount Δh in the range of the upper limit or below can make it possible to avoid a situation in which the base material 12 is broken.

As Δh becomes smaller, the planar shape of a stair, a groove, a convex portion, or the like constituting the three-dimensional pattern 40 can be finely expressed, and an elaborate three-dimensional shape can be processed. On the other hand, as Δh becomes larger, the processing time required for cutting can be shortened.

For example, from the viewpoint of achieving a three-dimensional pattern 40 in which an elaborate planar shape is processed, the cut amount Δh along the XY plane can be preferably 0.8 mm or less, and more preferably 0.6 mm or less.

Further, for example, from the viewpoint of shortening the processing time, the cut amount Δh along the XY plane can be preferably 0.1 mm or more, and more preferably 0.3 mm or more.

The hardness of the solid personal care product 10 manufactured by the cutting processing can be 0.1 N or more and 100 N or less, for instance.

The hardness of the solid personal care product 10 can be measured using a rheometer, a force gauge, a hardness meter, or the like. For example, the hardness of the solid personal care product 10 can be calculated by mounting a jig, which has a contact portion with the surface of the product of φ2 mm, on a rheometer (CR-100 manufactured by Sun Scientific Co., Ltd.) and measuring the load when the jig is pushed 1 mm from the surface of the product.

### Example 1

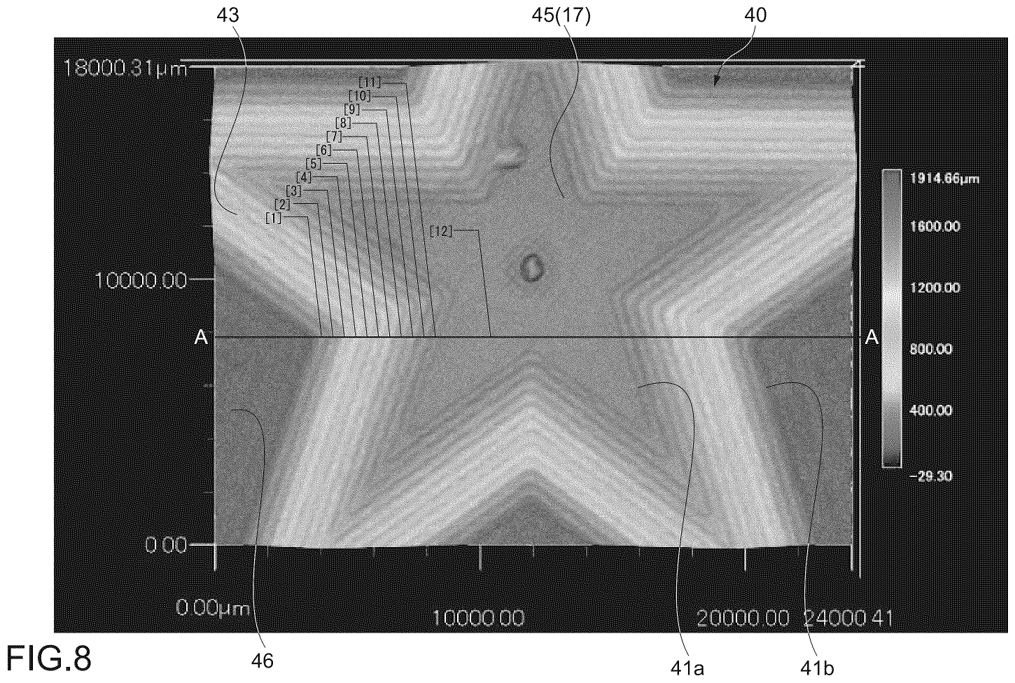

Example of the solid personal care product 10 having the three-dimensional pattern 40 formed by cutting will be described with reference to FIGS. 8, 9, and 10. In this Example, similarly to FIG. 2, a three-dimensional star mark having five vertices can be formed as the three-dimensional pattern 40. This mark can include a small star 41a and a large star 41b, according to one or more embodiments of the present disclosure. Of those, the small star 41a can be formed at a higher position.

Measurement positions [1] to [12] are illustrated on the line AA shown in FIG. 8. Of those, the measurement positions [1] to [11] can be set to be located at positions on the layer surfaces 44 of the respective stairs constituting the stepped portion 43. Further, the measurement position [12] can be set at the topmost portion 45 constituting the small star 41a. Note that the portion where the measurement position [12] is set can be an uncut pressed surface 17, according to one or more embodiments of the present disclosure.

In this Example, a cut amount Δv (stair width Δs) in the Z direction at the time of cutting out the stepped portion 43 was set to 150 μm. Further, the cut amount Δh in the XY directions was set to 0.5 mm. The cutting tool was a ball end mill with R of 0.5 mm, and a feed rate was set to 500 mm/min.

For a powder cosmetic serving as the base material, one obtained by mixing and pressing the following raw materials was used. The hardness calculated from the load obtained when a jig of φ2 mm was attached to a rheometer (CR-100 manufactured by Sun Scientific Co., Ltd.) and pushed 1 mm from the surface of the base material was 6.5 N.

### <Composition>

Oil: 11%
Pigment powder: 89%

For the pigment powder, a raw material made of talc, mica, titanium oxide, iron oxide, or the like was used. The mean particle size of the powder was 63.0 μm.

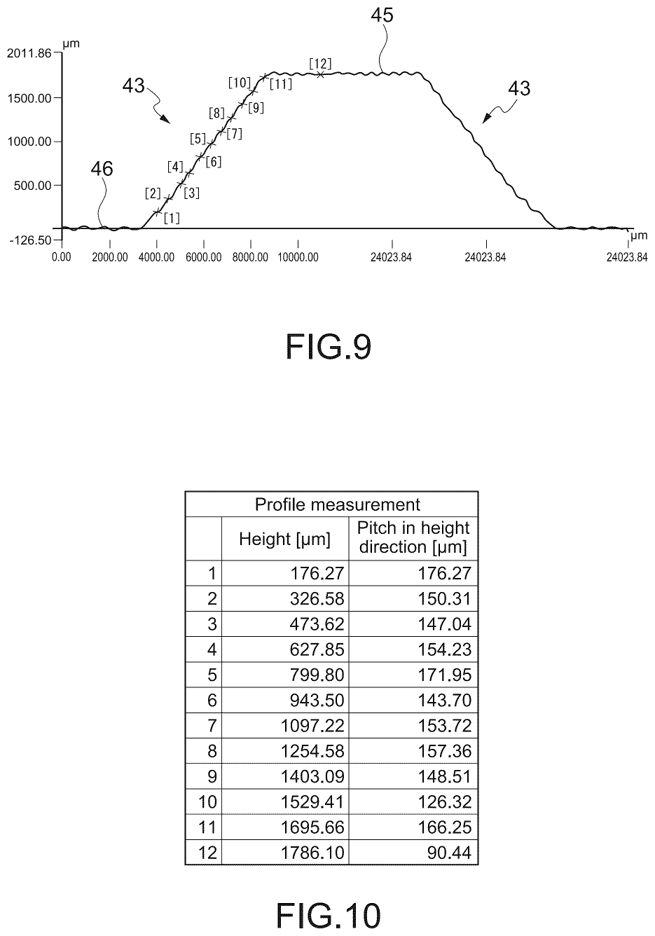

FIG. 9 shows the profile of the height (Z position) along the line AA shown in FIG. 8. Hereinafter, the Z position of each point relative to the bottommost portion 46 formed around the star 41b will be referred to as the height of each point.

FIG. 10 shows a height [μm] relative to the bottommost portion 46 and a pitch in the height direction [μm] at each of the measurement positions [1] to [12]. Here, the pitch in the height direction can be obtained by subtracting the height of the (i−1)-th measurement position from the height of the i-th measurement position and can represent a difference in height from a preceding measurement position, that is, a stair width of one stair.

As shown in FIG. 9, in this Example, a step-like structure corresponding to the layered stairs can be found in the profile of the stepped portion 43. Further, as shown in FIG. 10, when the pitch in the height direction between the measurement positions is viewed, the pitch can have a value close to 150 μm set as the cut amount Δv (stair width Δs), and it can be found that the stepped portion 43 can be properly formed.

Further, the height of the measurement position [12] that is the topmost portion 45 can be 1786.10 μm, and it can be found that a three-dimensional pattern 40 having a depth of 1 mm or more can be formed.

If the cut amount Δv is set to approximately 150 μm, the step of cutting in the Z-direction (first step) can be performed 12 times, for instance, so that a shape having the difference in height close to 1.8 mm can be cut out. This can make it possible to achieve an accurate three-dimensional pattern 40, for example, at a relatively high processing speed.

### Example 2

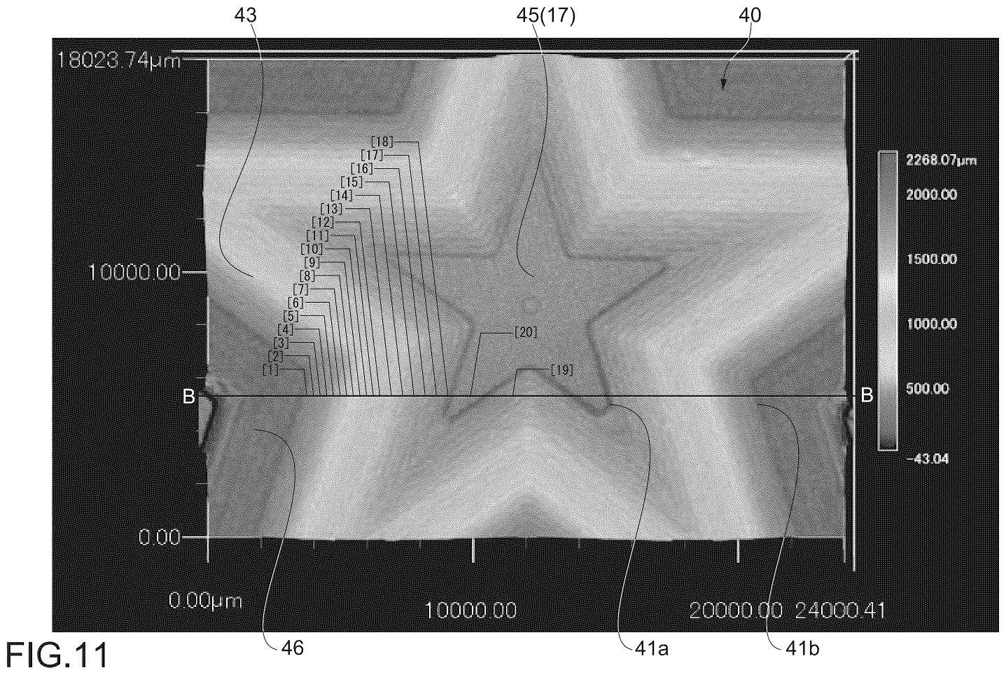

Another Example of the solid personal care product 10 having the three-dimensional pattern 40 formed by cutting will be described with reference to FIGS. 11, 12, and 13. In this Example, similarly to FIG. 2 and Example 1, a three-dimensional star mark having five vertices can be formed as the three-dimensional pattern 40.

Measurement positions [1] to [20] are illustrated on the line BB shown in FIG. 11. Of those, the measurement positions [1] to [18] can be set on a stepped portion 43 on the left side in the figure. Further, the measurement position [19] can be set on a stepped portion 43 formed on the right side of a topmost portion 45 connected with the stepped portion 43 on the left side in the figure. Further, the measurement position [20] can be set on the topmost portion 45 (pressed surface 17) constituting the small star 41a.

In this Example, in order to shorten the processing time, first, as a rough processing condition, the cut amount Δv (stair width Δs) in the Z direction at the time of cutting out the stepped portion 43 was set to 200 μm, and the cut amount Δh in the XY directions was set to 0.5 mm, to perform cutting. Subsequently, as a finishing processing condition, the cut amount Δv (stair width Δs) in the Z direction at the time of cutting out the stepped portion 43 was set to 100 μm, and the cut amount Δh in the XY directions was set to 0.5 mm, to perform cutting. The cutting tool was a ball end mill with R of 0.5 mm, and a feed rate was set to 1000 mm/min.

The powder cosmetic used as the base material was the same as that of Example 1.

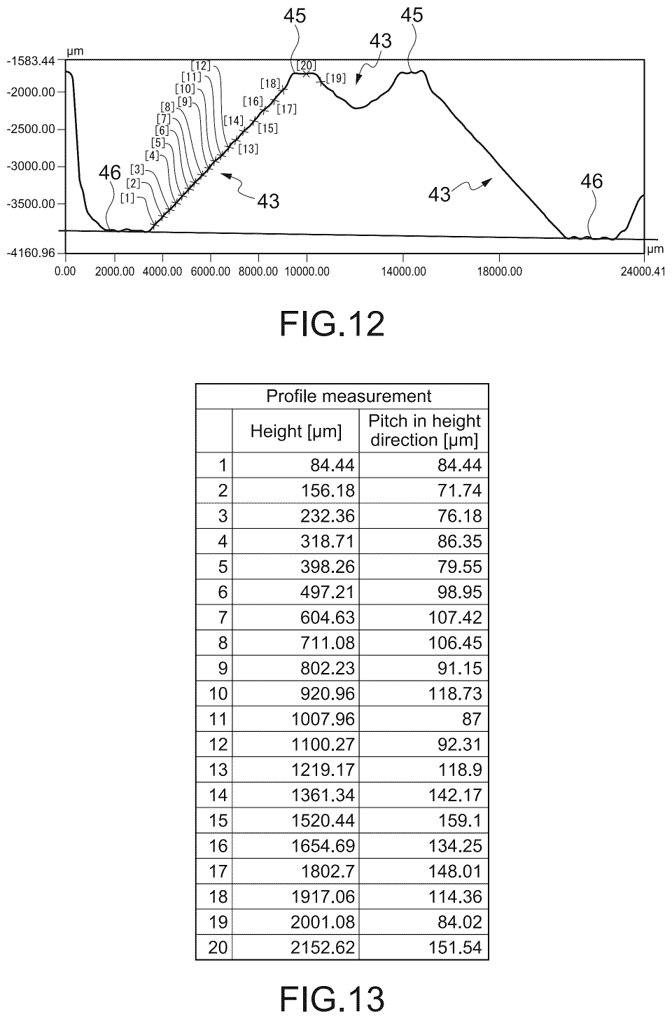

FIG. 12 shows the profile of the height (Z position) along the line BB shown in FIG. 11. Further, FIG. 13 shows a height [μm] relative to the bottommost portion 46 and a pitch in the height direction [μm] at each of the measurement positions [1] to [20].

As shown in FIG. 12, in this Example, the profile of the stepped portion 43 can constitute a smoother slope than that of Example shown in FIG. 9, for example. In other words, it can be found that the stepped portion 43 can be constituted as a smoother slope surface by using the stair width of 100 μm rather than the stair width of 150 μm.

Further, the height of the measurement position [20] that is the topmost portion 45 can be 2152.62 μm, and it can be found that a three-dimensional pattern 40 having a depth of 2 mm or more can be formed.

As described above, even when the cut amount Δv is set to approximately 100 μm, it can be possible to achieve a three-dimensional pattern 40 having a sufficient three-dimensional effect by providing a large difference in height. Further, since the cut amount Δv can be relatively small, it can be possible to accurately form a smooth slope, a curved surface, or the like.

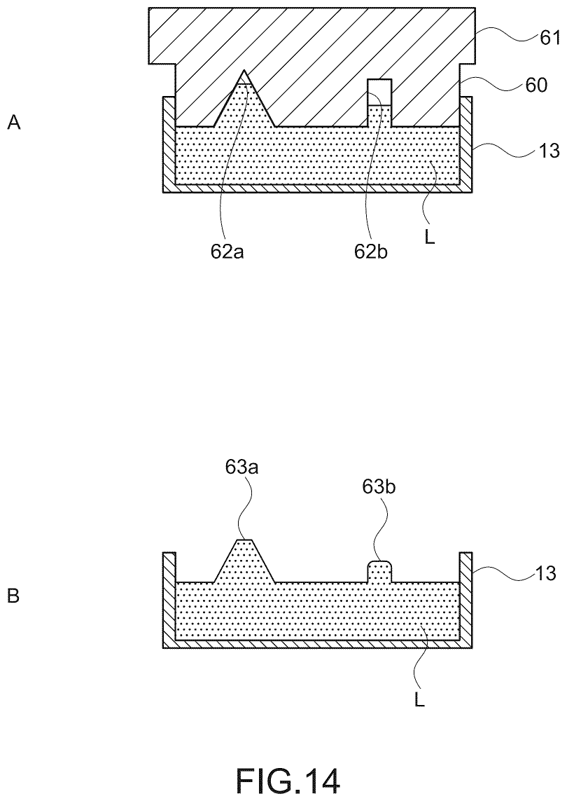

FIG. 14 illustrates an example of press molding using a mold as a comparative example. In FIG. 14A, a composition L housed in a container 13 is pressed by a press head 61 including a mold 60.

In the mold 60, a recess portion 62a having a triangular cross section and a recess portion 62b having a narrow width are formed. The mold 60 is pressed against the composition L in the container 13 by the press head 61. As a result, as shown in FIG. 14B, convex portions 63a and 63b shaped by the recess portions 62a and 62b provided in the mold 60 are formed on the composition L.

In the case of using the mold 60, there is a possibility that the transfer property of a corner portion is poor, and thus the corner portion is rounded or the corner portion is missing. For example, the tip may be chipped like the convex portion 63a of FIG. 14B, or the corner may be rounded like the convex portion 63b. Further, since the composition L is less likely to enter the recess portion 62b having a narrow width, it is difficult to accurately form the convex portion 63b having a narrow width.

As described above, it may be difficult to add decoration such as an elaborate pattern by using only the mold 60, and it may be difficult to achieve a design having a narrow width and a height.

The production of the mold 60 may be time-consuming and expensive, and there may be a possibility that the development period is prolonged and the initial investment is increased. Further, it may be difficult to deal with frequent design changes, customization of designs, and the like, and it may be difficult to deal with various types.

In addition, it can be conceivable that when a three-dimensional shape is formed by press molding using the mold 60, a density difference occurs in the base material 12 on which a three-dimensional shape is formed. For example, a portion strongly pressed by the mold 60 has a higher density than the other portions. Such a density difference may cause unevenness in the feeling of use of the product, such as the amount of powder cosmetic taken.

In the manufacturing method for the solid personal care product 10 according to this embodiment, the solid personal care product 10 having the three-dimensional pattern 40 can be manufactured by cutting the solid base material 12 provided on the target object 11 so as not to penetrate the base material 12. As the three-dimensional pattern 40, the stepped portion 43 including a plurality of layered stairs, each of which can have a stair width in the height direction of 0.001 mm or more and 1 mm or less, for instance, can be formed. Further, the base material 12 can be cut such that the difference in height between the topmost portion 45 and the bottommost portion 46 of the surface of the base material 12 can be 1 mm or more, for instance. This can make it possible to provide a solid personal care product 10 having an elaborate three-dimensional pattern 40 that is rich in a three-dimensional effect.

The solid base material 12 can be cut to form a three-dimensional pattern 40. Thus, it can be possible to achieve a design that is difficult to provide using a mold. For example, it can be possible to easily form a convex portion having a narrow width and a height, or a recess portion having a narrow width and a depth. Further, since corners may not be chipped or rounded, it can be possible to achieve an elaborate three-dimensional shape with high accuracy. This can make it possible to provide highly refined patterning to the surface of the base material 12 and to achieve an elaborate three-dimensional design.

In the cutting processing, the mold 60 or the like for forming a three-dimensional shape may not be necessary. This can make it possible to reduce the initial investment and shorten the development period (lead time).

Further, since the mold 60 may not be used, it can be easy to perform design changes, customization of designs, and the like. In addition, it can be possible to manufacture a single solid personal care product 10 having any three-dimensional pattern 40. For example, it can be possible to easily provide a solid personal care product 10 or the like having an original three-dimensional pattern 40 customized by an orderer.

Further, in the cutting processing, it can be possible to achieve a design rich in a three-dimensional effect, which can have a difference in height of 1 mm or more, for instance, without changing the density (porosity) of the base material 12, that is, without causing a difference in density of the cosmetic or the like. This can make it possible to provide a product having a three-dimensional design with height and having a stable feeling of use such as the amount of powder cosmetic taken.

### Other Embodiments

Hereinabove, one or more embodiments of the present disclosure have been described, but the present disclosure is not limited to the embodiment(s) described above and can be variously modified without departing from the gist of the present disclosure.

In the embodiment(s) described above, the configuration has been described, in which the difference in height between the topmost portion and the bottommost portion of the surface of the base material can be 1 mm or more, for instance, and a stepped portion including a plurality of layered stairs, each of which can have a stair width in the height direction of 0.001 mm or more and 1 mm or less, for instance, can be provided in the solid personal care product.

For example, it can be possible to provide a configuration in which the difference in height is less than 1 mm or a configuration in which the stepped portion is not provided.

A solid personal care product in which the difference in height is less than 1 mm, for instance, may be configured. In this case, a product in which a stepped portion can be formed on a solid base material on which a three-dimensional pattern is formed may be configured. Here, the stepped portion can be a portion formed as a three-dimensional pattern on the base material and including a plurality of layered stairs, each of which can have a stair width in the height direction of 0.001 mm or more and 1 mm or less, for instance.

In this configuration, it can be possible to achieve an elaborate three-dimensional pattern by the stepped portion.

A solid personal care product that does not include a stepped portion including a plurality of layered stairs may be configured. In this case, a product, which includes a solid base material on which a three-dimensional pattern can be formed, and in which the difference in height between the topmost portion and the bottommost portion of the surface of the base material can be 1 mm or more, for instance, may be formed.

In this configuration, since the difference in height can be 1 mm or more, for instance, a sufficient three-dimensional effect can be imparted to the design of the product.

In the above description, an example in which the base material housed in the container can be cut has been described, but the base material is not necessarily housed in the container. For example, for a solid personal care product that does not need a container, such as sloid soaps and solid bath additives, the solid base material itself may be cut without being placed in the container or the like. In this case as well, cutting may only need to be performed so as not to penetrate the base material.

In the above description, the case of using a cutting tool with a cutting blade, such as an end mill or a drill, has been described. However, the present disclosure is not limited to the above. For example, a laser cutting device that irradiates a base material with laser light to cut the base material, or the like may be used.

### Examples of Composition

Unless otherwise noted, the states (three states) of the substances to be descried below can be based on one atmospheric pressure and 20° C.

The composition can contain one kind or two or more kinds selected from solid substances such as powders and oils.

As such solid substances, for example, it can be preferable to contain powder used for normal cosmetic components such as a coloring pigment and an extender pigment.

Examples of the coloring pigment and the extender pigment can include an inorganic powder, an organic powder, and a compound powder of an inorganic powder and an organic powder.

Examples of the inorganic powder can include silicic acid, silicic anhydride, magnesium silicate, talc, sericite, mica, kaolin, red iron oxide, clay, bentonite, mica, titanium coated mica, bismuth oxychloride, zirconium oxide, magnesium oxide, titanium oxide, zinc oxide, aluminum oxide, calcium sulfate, barium sulfate, magnesium sulfate, calcium carbonate, magnesium carbonate, iron oxide, ultramarine, chromium oxide, chromium hydroxide, calamine, carbon black, boron nitride, and composites thereof.

Examples of the organic powder can include polyamide, nylon, polyester, polypropylene, polystyrene, polyurethane, vinyl resin, urea resin, phenol resin, fluoropolymer resin, silicone resin, acrylic resin, melamine resin, epoxy resin, polycarbonate resin, divinylbenzene-styrene copolymer, silk powder, cellulose, long-chain alkyl phosphoric acid metal salt, N-mono long-chain alkylacyl basic amino acid, and composites thereof.

Those extender pigments and coloring pigments can be colored or non-colored (e.g., white or substantially transparent), and can provide the composition or the skin with one or more among coloring, light diffraction, oil absorption, translucency, opacity, glossiness, a matte appearance, smoothness, and the like.

The content of the powder in the composition varies according to its purpose, but can be preferably 20 mass % or more, more preferably 30 mass % or more, and even more preferably 40 mass % or more, from the viewpoint of productivity such as drying.

The content of the powder in the composition can be preferably 85 mass % or less, more preferably 80 mass % or less, and even more preferably 70 mass % or less, from the viewpoint of productivity such as fluidity at the time of supply.

In such a range, a personal care product having a highly refined three-dimensional shape can be easily manufactured, and a good feeling of use when the product is used can be enhanced.

When a fluid composition is used, the oil that may be contained in the fluid can include one kind or two or more kinds selected from oils that are liquid at one atmospheric pressure and at 20° C. (hereinafter, also referred to as liquid oils) and oils that are solid at one atmospheric pressure and at 20° C. (hereinafter, also referred to as solid-state oils).

Examples of the liquid oils can include linear or branched hydrocarbon oils, plant oils, animal oils, ester oils, silicone oils, and high molecular alcohols.

Examples of the linear or branched hydrocarbon oils can include liquid paraffin and squalane. Examples of the plant oils can include a jojoba oil and an olive oil.

Examples of the animal oils can include liquid lanolin.

Examples of the ester oils can include monoalcohol fatty acid ester, and polyhydric alcohol fatty acid ester.

Examples of the silicone oils can include dimethylpolysiloxane, dimethylcyclopolysiloxane, methylphenylpolysiloxane, methylhydrogenpolysiloxane, and higher alcohol modified organopolysiloxane.

Examples of the high molecular alcohols can include polyethylene glycol.

Examples of the solid-state oils can include vaseline, cetanol, stearyl alcohol, and ceramide.

The content of the oil in the composition can vary according to its purpose, but can be preferably 0.5 mass % or more, more preferably 1 mass % or more, and even more preferably 1.5 mass % or more, as a total quantity.

The content of the oil in the composition can be preferably 30 mass % or less, more preferably 20 mass % or less, and even more preferably 15 mass % or less.

In such a range, favorable color developability and feel as a personal care product can be enhanced.

According to the type of a target personal care product, the composition can appropriately contain one kind or two or more kinds of components selected from thickeners, coating agents, surfactants, glucoses, polyhydric alcohols, water-soluble macromolecules, metal ion-sequestering agents, lower alcohols, amino acids, organic amines, pH control agents, skin conditioning agents, vitamins, antioxidants, flavoring substances, antiseptics, ultraviolet absorbers, ultraviolet scattering agents, and the like, within a range where the effects of the present disclosure are not impaired.

Examples of the ultraviolet absorbers can include one kind or two or more kinds selected from benzophenone derivatives and methoxycinnamate derivatives.

Examples of the benzophenone derivatives can include dihydroxybenzophenone, dihydroxydimethoxybenzophenone, hydroxymethoxybenzophenone sulfonate, and dihydroxydimethoxybenzophenone disulfonate.

Examples of the methoxycinnamate derivatives can include 2-ethylhexyl methoxycinnamate.

For the ultraviolet scattering agents, fine particles having a mean particle size of 0.1 μm or less can be used, as an example.

Examples of the ultraviolet scattering agents can include one kind or two or more kinds from zinc oxide, titanium oxide, and silica.

It may be preferable that the composition further include a liquid medium. The liquid medium can be a solvent to dissolve or disperse a cosmetic or a liquid that can be used as a dispersion medium.

In the case where the composition is in the form of slurry, the composition can be preferably a mixture containing at least a powder and a liquid medium. In the case where the composition is in the form of cosmetic slurry, the composition can be preferably a mixture containing at least a powder containing the pigment described above, an oil, and a liquid medium.

Examples of the above-mentioned liquid (liquid medium) can include a substance (volatile solvent) having volatility in a liquid state. Specifically, the liquid (liquid medium) can be preferably one kind or two or more kinds selected from water, alcohols, ketones, and hydrocarbons.

Examples of alcohols to be suitably used can include chain aliphatic monohydric alcohols with 1 to 6 carbon atoms, cyclic aliphatic monohydric alcohols with 3 to 6 carbon atoms, and aromatic monohydric alcohols. Specific examples of those alcohols include ethanol, isopropyl alcohol, butyl alcohol, phenylethyl alcohol, propanol, and pentanol.

Examples of ketones to be suitably used can include chain aliphatic ketones with 3 to 6 carbon atoms, cyclic aliphatic ketones with 3 to 6 carbon atoms, aromatic ketones with 8 to 10 carbon atoms. Specific examples of those ketones can include acetone, methyl ethyl ketone, methyl isobutyl ketone, cyclohexanone, and acetophenone.

Examples of hydrocarbons to be suitably used can include isoparaffin hydrocarbons, and specific examples thereof include an IP solvent.

In the case where the composition includes the liquid medium, the content of the liquid medium in the composition can vary according to its purpose, but can be preferably 20 mass % or more, more preferably 30 mass % or more, and even more preferably 40 mass % or more, as a total quantity.

The content of the liquid medium in the composition can be preferably 70 mass % or less, more preferably 60 mass % or less, and even more preferably 50 mass % or less.

In such a range, uniform dispersibility of the constituent materials of the composition can be enhanced, and the handleability can be enhanced.

Embodiments of the disclosed subject matter can also be as set forth according to the following parentheticals.

(1) A manufacturing method for a solid personal care product having a three dimensional pattern, comprising: a cutting process of cutting a solid base material provided on a target object such that the base material is not penetrated, wherein in the cutting process, the base material is cut such that a stepped portion is formed, the stepped portion including a plurality of layered stairs each having a stair width in a height direction of 0.001 mm or more and 1 mm or less, and a difference in height between a topmost portion and a bottommost portion of a surface of the base material is 1 mm or more.

(2) The manufacturing method for a solid personal care product according to (1), wherein in the cutting process, the base material is cut by relatively moving the target object and a cutting tool, which comes into contact with the base material to cut the base material.

(3) The manufacturing method for a solid personal care product according to (1) or (2), wherein in the cutting process, a first step of moving the cutting tool by a predetermined amount along the height direction and a second step of moving the cutting tool in the plane direction orthogonal to the height direction are alternately repeated to form the stepped portion.

(4) The manufacturing method for a solid personal care product according to any one of (1) to (3), wherein the stepped portion includes a plurality of layer surfaces orthogonal to the height direction, and in the cutting process, the layer surfaces are sequentially formed from a higher position in the height direction.

(5) The manufacturing method for a solid personal care product according to any one of (1) to (4), wherein a cut amount with respect to the base material in the height direction by the cutting tool is 0.001 mm or more and 1 mm or less.

(6) The manufacturing method for a solid personal care product according to any one of (1) to (5), wherein a cut amount with respect to the base material in the plane direction orthogonal to the height direction by the cutting tool is 0.001 mm or more and 1 mm or less.

(7) The manufacturing method for a solid personal care product according to any one of (1) to (6), wherein a cut amount with respect to the base material in the plane direction orthogonal to the height direction by the cutting tool is 0.8 mm or less.

(8) The manufacturing method for a solid personal care product according to any one of (1) to (7), wherein a cut amount with respect to the base material in the plane direction orthogonal to the height direction by the cutting tool is 0.1 mm or more.

(9) The manufacturing method for a solid personal care product according to any one of (1) to (8), wherein the cutting tool includes a rotary blade, and the rotary blade has a blade diameter of 0.1 mm or more and 5 mm or less.

(10) The manufacturing method for a solid personal care product according to any one of (1) to (9), wherein the cutting tool includes a rotary blade, and the rotary blade has a blade diameter of 0.1 mm or more and 2 mm or less.

(11) The manufacturing method for a solid personal care product according to any one of (1) to (10), wherein the cutting tool includes a rotary blade, and the rotary blade has a blade diameter of 2 mm or more and 5 mm or less.

(12) The manufacturing method for a solid personal care product according to any one of (1) to (11), wherein the base material is provided with the three dimensional pattern such that at least a part of the surface of the base material cut in the cutting process is included in a surface of a final product.

(13) The manufacturing method for a solid personal care product according to any one of (1) to (12), wherein the base material is a powder cosmetic pressed on the target object.

(14) The manufacturing method for a solid personal care product according to any one of (1) to (13), wherein the base material is pressed such that a surface of the powder cosmetic exposed on the target object is a flat surface.

(15) The manufacturing method for a solid personal care product according to any one of (1) to (14), wherein a cutter that performs the cutting process includes: a stage on which the target object is placed, a cutting tool that cuts the target object, a driver that drives the cutting tool, a support that supports the driver with respect to the stage, and a cutting controller that controls an operation of the cutter.

(16) A solid personal care product having a three dimensional pattern, comprising: a solid base material on which the three dimensional pattern is formed; and a stepped portion that is formed as the three dimensional pattern on the base material and includes a plurality of layered stairs each having a stair width in a height direction of 0.001 mm or more and 1 mm or less.

(17) The solid personal care product having a three dimensional pattern according to (16), wherein a difference in height between a topmost portion and a bottommost portion of a surface of the base material is 1 mm or more.

(18) The solid personal care product having a three dimensional pattern according to (16) or (17), wherein the base material has a hardness of 0.1 N or more and 100 N or less.

(19) The solid personal care product having a three dimensional pattern according to any one of (16) to (18), wherein the base material includes powder, and the powder has a mean particle size of 0.01 μm or more and 1 mm or less.

(20) A solid personal care product having a three dimensional pattern, comprising: a solid base material on which the three dimensional pattern is formed, wherein the three dimensional pattern has a stepped portion that is formed as the three dimensional pattern on the solid base material and that includes a plurality of layered stairs each having a stair width in a height direction of 0.001 mm or more and 1 mm or less, a difference in height between a topmost portion and a bottommost portion of a surface of the solid base material is 1 mm or more.

(21) The manufacturing method for a solid personal care product according to any one of (1) to (15), wherein the stair width in the height direction of one or more of the layered steps is restricted according to a first restriction range of 0.2 mm or less, and/or a stair width in a plane direction orthogonal to the height direction of each of said one or more layered steps is restricted according to a second restriction range of 0.001 mm or more and/or 1 mm or less. (22) The solid personal care product having a three dimensional pattern according to any one of (16) to (19), wherein the stair width in the height direction of one or more of the layered steps is restricted according to a first restriction range of 0.2 mm or less, and/or a stair width in a plane direction orthogonal to the height direction of each of said one or more layered steps is restricted according to a second restriction range of 0.001 mm or more and/or 1 mm or less.

(23) The solid personal care product having a three dimensional pattern according to (20), wherein the stair width in the height direction of one or more of the layered steps is restricted according to a first restriction range of 0.2 mm or less, and/or a stair width in a plane direction orthogonal to the height direction of each of said one or more layered steps is restricted according to a second restriction range of 0.001 mm or more and/or 1 mm or less.

### REFERENCE SIGNS LIST

10 solid personal care product
11 target object
12 base material
33 cutting tool
43 stepped portion
45 topmost portion
46 bottommost portion

1. A manufacturing method for a solid personal care product having a three dimensional pattern, comprising:
a cutting process of cutting a solid base material provided on a target object such that the base material is not penetrated, wherein
in the cutting process, the base material is cut such that a stepped portion is formed, the stepped portion including a plurality of layered stairs each having a stair width in a height direction of 0.001 mm or more and 1 mm or less, and a difference in height between a topmost portion and a bottommost portion of a surface of the base material is 1 mm or more.

2. The manufacturing method for a solid personal care product according to claim 1, wherein
in the cutting process, the base material is cut by relatively moving the target object and a cutting tool, which comes into contact with the base material to cut the base material.

3. The manufacturing method for a solid personal care product according to claim 2, wherein
in the cutting process, a first step of moving the cutting tool by a predetermined amount along the height direction and a second step of moving the cutting tool in a plane direction orthogonal to the height direction are alternately repeated to form the stepped portion.

4. The manufacturing method for a solid personal care product according to claim 2, wherein
the stepped portion includes a plurality of layer surfaces orthogonal to the height direction, and
in the cutting process, the layer surfaces are sequentially formed from a higher position in the height direction.

5. The manufacturing method for a solid personal care product according to claim 2, wherein
a cut amount with respect to the base material in the height direction by the cutting tool is 0.001 mm or more and 1 mm or less, and
a cut amount with respect to the base material in a plane direction orthogonal to the height direction by the cutting tool is 0.001 mm or more and 1 mm or less.

6. The manufacturing method for a solid personal care product according to claim 2, wherein
a cut amount with respect to the base material in the plane direction orthogonal to the height direction by the cutting tool is 0.8 mm or less, and
a cut amount with respect to the base material in the plane direction orthogonal to the height direction by the cutting tool is 0.1 mm or more.

7. The manufacturing method for a solid personal care product according to claim 2, wherein
the cutting tool includes a rotary blade, and
the rotary blade has a blade diameter of 0.1 mm or more and 5 mm or less.

8. The manufacturing method for a solid personal care product according to claim 2, wherein
the cutting tool includes a rotary blade, and
the rotary blade has a blade diameter of 0.1 mm or more and 2 mm or less.

9. The manufacturing method for a solid personal care product according to claim 2, wherein
the cutting tool includes a rotary blade, and
the rotary blade has a blade diameter of 2 mm or more and 5 mm or less.

10. The manufacturing method for a solid personal care product according to claim 1, wherein
the base material is provided with the three dimensional pattern such that at least a part of the surface of the base material cut in the cutting process is included in a surface of a final product.

11. The manufacturing method for a solid personal care product according to claim 1, wherein
the base material is a powder cosmetic pressed on the target object.

12. The manufacturing method for a solid personal care product according to claim 11, wherein
the base material is pressed such that a surface of the powder cosmetic exposed on the target object is a flat surface.

13. The manufacturing method for a solid personal care product according to claim 1, wherein
a cutter that performs the cutting process includes:
a stage on which the target object is placed,
a cutting tool that cuts the target object,
a driver that drives the cutting tool,
a support that supports the driver with respect to the stage, and
a cutting controller that controls an operation of the cutter.

14. The manufacturing method for a solid personal care product according to claim 1, wherein
the stair width in the height direction of one or more of the layered steps is restricted according to a first restriction range of 0.2 mm or less, and
a stair width in a plane direction orthogonal to the height direction of each of said one or more layered steps is restricted according to a second restriction range of 0.001 mm or more and 1 mm or less.

15. A solid personal care product having a three dimensional pattern, comprising:
a solid base material on which the three dimensional pattern is formed; and
a stepped portion that is formed as the three dimensional pattern on the base material and includes a plurality of layered stairs each having a stair width in a height direction of 0.001 mm or more and 1 mm or less.

16. The solid personal care product having a three dimensional pattern according to claim 15, wherein
a difference in height between a topmost portion and a bottommost portion of a surface of the base material is 1 mm or more.

17. The solid personal care product having a three dimensional pattern according to claim 15, wherein
the base material has a hardness of 0.1 N or more and 100 N or less.

18. The solid personal care product having a three dimensional pattern according to claim 15, wherein
the base material includes powder, and
the powder has a mean particle size of 0.01 μm or more and 1 mm or less.

19. The solid personal care product having a three dimensional pattern according to claim 15, wherein
the stair width in the height direction of one or more of the layered steps is restricted according to a first restriction range of 0.2 mm or less, and/or
a stair width in a plane direction orthogonal to the height direction of each of said one or more layered steps is restricted according to a second restriction range of 0.001 mm or more and/or 1 mm or less.

20. A solid personal care product having a three dimensional pattern, comprising:
a solid base material on which the three dimensional pattern is formed, wherein
the three dimensional pattern has a stepped portion that is formed as the three dimensional pattern on the solid base material and that includes a plurality of layered stairs each having a stair width in a height direction of 0.001 mm or more and 1 mm or less,
a difference in height between a topmost portion and a bottommost portion of a surface of the solid base material is 1 mm or more.
